(19) United States
(12) Patent Application Publication
(10) Pub. No.: US 2017/0204032 A1
(43) Pub. Date: Jul. 20, 2017

(54) PROCESS FOR PREPARING BRANCHED ALLYL COMPOUNDS

(71) Applicant: South University of Science and Technology of China, Shenzhen (CN)

(72) Inventor: Chun-Yu Ho, Hong Kong (CN)

(73) Assignee: South University of Science and Technology of China, Guangdong (CN)

(21) Appl. No.: 15/326,791

(22) PCT Filed: Jul. 23, 2014
(86) PCT No.: PCT/CN2014/082861

§ 371 Date: Jan. 17, 2017

(51) Int. Cl. C07C 41/30; B01J 31/22

(52) U.S. Cl. CPC C07C 41/30; B01J 31/2265

(57) ABSTRACT

Disclosed are a process for preparing branched allyl compounds with an unsymmetrical 1,1-disubstituted alkene, and compounds prepared therewith.

### TECHNICAL FIELD

The present application is directed to organic synthesis chemistry, in particular to a process for preparing branched allyl compounds.

### BACKGROUND

Synthesis of new branched allyl compounds, particularly branched allyl compounds with an unsymmetrical 1,1-disubstituted alkene general structure is one of the most important aspects in organic synthesis chemistry, since branched allyl compounds have been found in broad applications due to their unique properties such as electrical conductivity, magnetism, and chemical reactivity.

Methods for preparing branched allyl compounds with an unsymmetrical 1,1-disubstituted alkene general structure in the art may generally rely on expensive starting materials. Most of the methods are stoichiometric in nature or suffer from significant waste disposal problems in a large scale (e.g. phosphine oxide, arylsulfonate, titanium/aluminum salt, silyl ether and halides) or substrate availability.

There are only very limited availability and choices of 1,1-disubstituted alkenes and branched allyl compounds on the market, mainly constrained by the availability of natural products, petroleum cracking and selective dehydrogenation of several alkanes. According to the Sigma-Aldrich product catalogue, there are only around twenty 1,1-disubstituted alkenes available in stock (a very low amount compared to aromatic alkenes and alpha-alkenes, http://www.sigmaldrich.com/chemistry/chemistry-products.html?TablePage=62744-29).

Synthesis of branched allyl compounds with an unsymmetrical 1,1-disubstituted alkene general structure from linear allyl or vinyl compounds or α-olefins may suffer from side reactions such as olefin isomerization/oligomerization, self-dimerization, and hydrogenative dimerization, resulting in a mixture of regioisomers and a significant amount of toxic transition metal waste.

### SUMMARY

The present application is to change the typically observed reactivity pattern of vinyl hetero-substituted compounds and α-olefins towards transition metal complex, and to control the regioselectivity of a carbon-carbon bond forming reaction between two alkenes, strongly favoring the production of branched allyl compounds with an 1,1-disubstituted alkene general structure in a tail-to-tail fashion.

The present application is also to provide a cost effective and environmentally friendly way to fulfill the increasing demand of both branched allyl, vinyl hetero-substituted compounds and 1,1-disubstituted alkenes.

The present application converts relatively unreactive alkenes to more reactive ones. With this technology, desired products can be provided with conventional olefins through branched allyl compounds functionalization.

In one aspect, the present application provides a process for preparing a compound of formula (III), comprising reacting a compound of formula (I) with a compound of formula (II) in the presence of a transition metal catalyst or a precursor thereof,

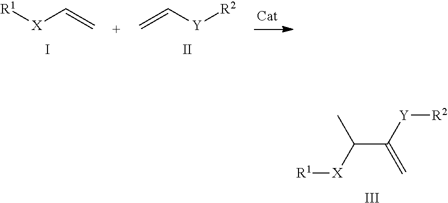

wherein,

X is independently selected from the atomic group consisting of Group 13 and Group 15-17 on the Periodic Table,

Y is independently selected from the atomic group consisting of Group 13 to 17 on the Periodic Table,

R¹ and R² are each independently selected from the group consisting of H, alkyl, alkenyl, aryl, arylalkyl, hydroxyl, alkoxyl, aroxyl, carbonyl, phosphonyl, halogen, amino, alkylamino, arylamino, mercapto, alkylthio and arylthio.

In another aspect, the present application is directed to a compound of formula (III)

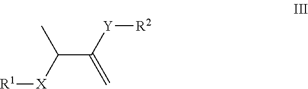

wherein,

X is selected from the atomic group consisting of Group 13 and Group 15-17 on the Periodic Table,

Y is independently selected from the atomic group consisting of Group 13 to 17 on the Periodic Table,

R¹ and R² are each independently selected from the group consisting of H, alkyl, alkenyl, aryl, arylalkyl, hydroxyl, alkoxyl, aroxyl, carbonyl, phosphonyl, halogen, amino, alkylamino, arylamino, mercapto, alkylthio and arylthio.

### BRIEF DESCRIPTION OF THE FIGURES

FIGS. 1A and 1B show ¹H- and ¹³C-NMR spectra of the compound (III).

### DETAILED DESCRIPTION OF THE PREFERRED EMBODIMENTS

### Definitions

In the following description, certain specific details are included to facilitate a thorough understanding of various disclosed embodiments. One skilled in the relevant art, however, will recognize that embodiments may be practiced without one or more of these specific details, or with other methods, components, materials, etc.

Unless the context requires otherwise, throughout the specification and claims which follow, the word “comprise” and variations thereof, such as “comprises” and “comprising”, are to be construed in an open, inclusive sense, which is as “including, but not limited to”.

Reference throughout this specification to “one embodiment”, or “an embodiment”, or “in another embodiment”, or “some embodiments”, or “in some embodiments” means that a particular referent feature, structure, or characteristic described in connection with the embodiment is included in at least one embodiment. Thus, the appearance of the phrases “in one embodiment”, or “in an embodiment”, or “in another embodiment”, or “in some embodiments” in various places throughout this specification are not necessarily all referring to the same embodiment. Furthermore, the particular features, structures, or characteristics may be combined in any suitable manner in one or more embodiments.

It should be noted that, as used in this specification and the appended claims, the singular forms “a”, “an”, and “the” include plural referents unless the content clearly indicates otherwise. In the present application, the use of “or” means “and/or” unless specifically stated otherwise.

Certain chemical groups named herein are preceded by a shorthand notation indicating the total number of carbon atoms that are to be found in the indicated chemical group. For example, C₇-C₁₂ alkyl describes an alkyl group, as defined below, having a total of 7 to 12 carbon atoms. The total number of carbons in the shorthand notation does not include carbons that may exist in substituents of the group described.

As used herein, “Cₘ to Cₙ” or “C_{m to n}” in which “m” and “n” are integers refers to the number of carbon atoms in an alkyl or alkenyl group or the number of carbon atoms in the ring of a cycloalkyl or cycloalkenyl group. That is, the alkyl, alkenyl, ring of the cycloalkyl or ring of the cycloalkenyl can contain from “m” to “n”, inclusively, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “m” and “n” are designated with regard to an alkyl, alkenyl, cycloalkyl or cycloalkenyl group, the broadest range described in these definitions is to be assumed.

Accordingly, as used in the specification and appended claims, unless specified to the contrary, the following terms have the meaning indicated:

The term “alkyl” as used herein alone or as part of a group means any unbranched or branched, substituted or unsubstituted, saturated hydrocarbon group. The alkyl moiety may be a branched or straight chain. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl.

The alkyl group may be substituted or unsubstituted. When substituted, the substituent group may be one or more groups individually and independently selected from substituted or unsubstituted cycloalkyl, substituted or unsubstituted cycloalkenyl, substituted or unsubstituted aryl, substituted or unsubstituted heteroaryl, substituted or unsubstituted heteroaryloxy, heterocyclyl, heterocyclyloxy, heteroalicyclyl, hydroxy, substituted or unsubstituted alkoxy, substituted or unsubstituted aryloxy, acyl, thiol, substituted or unsubstituted thioalkoxy, alkylthio, arylthio, cyano, halo, carbonyl, thiocarbonyl, acylalkyl, acylamino, acyloxy, aminoacyl, aminoacyloxy, oxyacylamino, keto, thioketo, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and substituted or unsubstituted amino, including mono- and di-substituted amino groups, and the protected derivatives thereof, hydroxyamino, alkoxyamino, —SO-alkyl, —SO-substituted alkyl, —SO-aryl, —SO-heteroaryl, —SO₂-alkyl, —SO₂-substituted alkyl, —SO₂-aryl and —SO₂-heteroaryl. Typical alkyl groups include, but are in no way limited to, methyl, ethyl, propyl, isopropyl, butyl, isobutyl, tertiary butyl, pentyl, hexyl, and the like.

The term “alkenyl” as used herein alone or as part of a group refers to a straight or branched hydrocarbon chain group consisting solely of carbon and hydrogen atoms, containing at least one double bond, having from two to twelve carbon atoms, preferably two to eight carbon atoms and which is attached to the rest of the molecule by a single bond, e.g., ethenyl, propenyl, butenyl, pentenyl, penta-1,4-dienyl, and the like.

The term “cycloalkyl” as used herein alone or as part of a group refers to a completely saturated (no double bonds) mono- or multi-cyclic hydrocarbon ring system. When composed of two or more rings, the rings may be joined together in a fused, bridged or spiro-connected fashion. Cycloalkyl groups of the present application may range from C₃ to C₁₀. In other embodiments, it may range from C₃ to C₆. A cycloalkyl group may be unsubstituted or substituted. Typical cycloalkyl groups include, but are not limited to, cyclopropyl, cyclobutyl, cyclopentyl, cyclohexyl, and the like. If substituted, the substituent(s) may be an alkyl or selected from those indicated above with regard to substitution of an alkyl group unless otherwise indicated.

The term “cycloalkenyl” as used herein alone or as part of a group refers to a cycloalkyl group that contains one or more double bonds in the ring although, if there is more than one, they cannot form a fully delocalized pi-electron system in the ring (otherwise the group would be “aryl”, as defined herein). When composed of two or more rings, the rings may be connected together in a fused, bridged or spiro-connected fashion. A cycloalkenyl group of the present application may be unsubstituted or substituted. When substituted, the substituent(s) may be an alkyl or selected from the groups disclosed above with regard to alkyl group substitution unless otherwise indicated. The number of carbon atoms in the cycloalkenyl may be in the range of 3 to 10.

The term “carbonyl” as used herein alone or as part of a group refers to the group —(C═O).

The term “alkoxy” as used herein alone or as part of a group refers to any unbranched, or branched, substituted or unsubstituted, saturated or unsaturated ether, with C₁-C₆ unbranched, saturated, unsubstituted ethers being preferred, with methoxy and ethoxy being more preferred.

The term “alkylamino” as used herein alone or as part of a group refers to the group —NH-alkyl.

The term “halo” or “halogen” as used herein alone or as part of a group refers to bromo, chloro, fluoro or iodo.

The term “heterocyclyl” as used herein alone or as part of a group is intended to mean three-, four-, five-, six-, seven-, and eight- or more membered rings wherein carbon atoms together with from 1 to 3 heteroatoms constitute the ring. A heterocyclyl can optionally contain one or more unsaturated bonds situated in such a way, however, that an aromatic pi-electron system does not arise. The heteroatoms are independently selected from oxygen, sulfur, and nitrogen.

A heterocyclyl can further contain one or more carbonyl or thiocarbonyl functionalities, so as to make the definition include oxo-systems and thio-systems such as lactams, lactones, cyclic imides, cyclic thioimides, cyclic carbamates, and the like.

Heterocyclyl rings can optionally be fused ring systems containing two or more rings wherein at least one atom is shared between two or more rings to form bicyclic or tricyclic structures. In some embodiments, such fused ring systems are formed by a bridging moiety between two atoms of a heterocyclyl.

Heterocyclyl rings can optionally also be fused to aryl rings, such that the definition includes bicyclic structures. Typically such fused heterocyclyl groups share one bond with an optionally substituted benzene ring. Examples of benzo-fused heterocyclyl groups include, but are not limited to, benzimidazolidinone, tetrahydroquinoline, and methylenedioxybenzene ring structures.

Some examples of “heterocyclyls” include, but are not limited to, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 1,3-oxathiolane, and an azabicyclo system such as azabicyclo[3.2.1]octyl (tropane). Binding to the heterocycle can be at the position of a heteroatom or via a carbon atom of the heterocycle, or, for benzo-fused derivatives, via a carbon of the benzenoid ring.

The term “aromatic” as used herein refers to an aromatic group which has at least one ring having a conjugated pi electron system and includes both carbocyclic aryl (e.g., phenyl) and heterocyclic aryl groups (e.g., pyridine). The term includes monocyclic or fused-ring polycyclic (i.e., rings which share adjacent pairs of carbon atoms) groups.

The term “carbocyclic” as used herein, refers to a compound which contains one or more covalently closed ring structures, and that the atoms forming the backbone of the ring are all carbon atoms. The term thus distinguishes carbocyclic from heterocyclic rings in which the ring backbone contains at least one atom which is different from carbon. The term “heteroaromatic” as used herein, refers to an aromatic group which contains at least one heterocyclic ring.

The term “aryl” as used herein alone or as part of a group is intended to mean a carbocyclic aromatic ring or ring system. Moreover, the term “aryl” includes fused ring systems wherein at least two aryl rings, or at least one aryl and at least one C₃₋₈-cycloalkyl share at least one chemical bond. Some examples of “aryl” rings include optionally substituted phenyl, naphthalenyl, phenanthrenyl, anthracenyl, tetralinyl, fluorenyl, indenyl, and indanyl.

The term “aryl” relates to aromatic, including, for example, benzenoid groups, connected via one of the ring-forming carbon atoms, and optionally carrying one or more substituents selected from heterocyclyl, heteroaryl, halo, hydroxy, amino, cyano, nitro, alkylamido, acyl, C₁₋₆-alkoxy, C₁₋₆-alkyl, C₁₋₆-hydroxyalkyl, C₁₋₆-aminoalkyl, C₁₋₆-alkylamino, alkylsulfenyl, alkylsulfinyl, alkylsulfonyl, sulfamoyl, or trifluoromethyl. The aryl group can be substituted at the para and/or meta positions. In other embodiments, the aryl group can be substituted at the ortho position. Representative examples of aryl groups include, but are not limited to, phenyl, 3-halophenyl, 4-hydroxyphenyl, 3-aminophenyl, 4-aminophenyl, 3-methylphenyl, 4-methoxyphenyl, 4-trifluoromethylphenyl, 4-cyanophenyl, dimethylphenyl, naphthyl, hydroxynaphthyl, 4-pyrazolylphenyl, 4-triazolylphenyl, and 4-(2-oxopyrrolidin-1-yl)phenyl.

The term “arylalkyl” or “aralkyl” as used herein alone or as part of a group which are used synonymously and interchangeably refers to an aryl group covalently bonded to an alkyl group, as defined herein. A “phenylalkyl” is a species of an aralkyl group, and refers to a phenyl ring covalently bonded to an alkyl group as defined herein. Examples of phenylalkyl groups include, but are not limited to, benzyl, 2-phenylethyl, 1-phenylpropyl, 3-phenylamyl and 3-phenyl-2-methylpropyl. Presently preferred phenylalkyl groups are those wherein the phenyl group is covalently bonded to one of the presently preferred alkyl groups. A phenyl alkyl group of the present application may be unsubstituted or substituted. Examples of substituted phenylalkyl groups include, but are not limited to, 2-phenyl-1-chloroethyl, 2-(4-methoxyphenyl)ethyl, and 5-phenyl-3-oxo-pent-1-yl.

The term “heteroaryl” as used herein alone or as part of a group is intended to mean a heterocyclic aromatic group where one or more carbon atoms in an aromatic ring have been replaced with one or more heteroatoms selected from the group comprising nitrogen, sulfur, and oxygen.

Furthermore, in the present context, the term “heteroaryl” comprises fused ring systems wherein at least one aryl ring and at least one heteroaryl ring, at least two heteroaryl rings, at least one heteroaryl ring and at least one heterocyclyl ring, or at least one heteroaryl ring and at least one cycloalkyl ring share at least one chemical bond.

The term “heteroaryl” is understood to relate to aromatic, C₃₋₈ cyclic groups further containing one oxygen or sulfur atom or up to four nitrogen atoms, or a combination of one oxygen or sulfur atom with up to two nitrogen atoms, and their substituted as well as benzo- and pyrido-fused derivatives, for example, connected via one of the ring-forming carbon atoms. Heteroaryl groups can carry one or more substituents selected from halo, hydroxy, amino, cyano, nitro, alkylamido, acyl, C₁₋₆-alkoxy, C₁₋₆-alkyl, C₁₋₆-hydroxyalkyl, C₁₋₆-aminoalkyl, C₁₋₆-alkylamino, alkylsulfenyl, alkylsulfinyl, alkylsulfonyl, sulfamoyl, or trifluoromethyl. In some embodiments, heteroaryl groups can be five- and six-membered aromatic heterocyclic systems carrying 0, 1, or 2 substituents, which can be the same as or different from one another, selected from the list above.

Representative examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, imidazole, benzimidazole, pyrazole, indazole, tetrazole, quinoline, 1,2,3-oxadiazole, 1,2,4-thiadiazole, triazole, benzotriazole, pteridine, phenoxazole, oxadiazole, benzopyrazole, quinolizine, cinnoline, phthalazine, quinazoline, and quinoxaline. In some embodiments, the substituents are halo, hydroxy, cyano, O—C₁₋₆-alkyl, C₁₋₆-alkyl, hydroxy-C₁₋₆-alkyl, and amino-C₁₋₆-alkyl.

The term “phenyl” as used herein alone or as part of a group refers to a six-membered aryl group. A phenyl group may be unsubstituted or substituted. When substituted the substituent(s) is(are) one or more, preferably one or two, group(s) independently selected from the group consisting of halogen, hydroxy, protected hydroxy, cyano, nitro, alkyl, alkoxy, acyl, acyloxy, carboxy, protected carboxy, carboxymethyl, protected carboxymethyl, hydroxymethyl, protected N-alkylcarboxamide, protected N-alkylcarboxamide, N,N-dialkylcarboxamide, trifluoromethyl, N-alkylsulfonylamino, N-(phenylsulfonyl)amino and phenyl (resulting in the formation of a biphenyl group).

Examples of substituted phenyl groups include, but are not limited to, 2-, 3- or 4-chlorophenyl, 2,6-dichlorophenyl, 2-, 3- or 4-hydroxyphenyl, 2,4-dihydroxyphenyl, the protected-hydroxy derivatives thereof.

The term “mercapto” as used herein refers to a group of formula “—SH”.

The term “alkylthio” as used herein alone or as part of a group refers to an “alkyl-S—” group, with alkyl as defined above. Examples of alkylthio group include, but are not limited to, methylthio, ethylthio, n-propylthio, isopropylthio, n-butylthio and t-butylthio.

The term “arylthio” as used herein alone or as part of a group refers to an “aryl-S—” group, with aryl as defined above. Examples of arylthio group include, but are not limited to, phenylthio, naphthylthio, and anthracylthio.

The term “alkylsulfinyl” as used herein alone or as part of a group refers to an “alkyl-SO—” group, with alkyl as defined above. Examples of alkylsulfinyl groups include, but are not limited to, methylsulfinyl, ethylsulfinyl, n-propylsulfinyl, isopropylsulfinyl, n-butylsulfinyl and sec-butylsulfinyl.

The term “alkylsulfonyl” as used herein alone or as part of a group refers to an “alkyl-SO₂—” group. Examples of alkylsulfonyl groups include, but are not limited to, methylsulfonyl, ethylsulfonyl, n-butylsulfonyl, and t-butylsulfonyl.

The terms “phenylthio”, “phenylsulfinyl”, and “phenylsulfonyl” as used herein alone or as part of a group refer to a “phenyl-S—”, “phenyl-SO—”, and “phenyl-SO₂—” group, phenyl as defined herein.

The term “amine” as used herein refers to a compound that comprises an amino group. The term “amino” as used herein alone or as part of a group refers to the —NH₂ radical.

The term “cyano” as used herein alone or as part of a group refers to the —CN radical.

The term “hydroxy” as used herein alone or as part of a group refers to the —OH radical.

The term “imine” as used herein refers to a compound that comprises an imino group. The term “imino” as used herein alone or as part of a group refers to the ═NH substituent.

The term “nitro” as used herein alone or as part of a group refers to the —NO₂ radical.

The term “oxo” as used herein alone or as part of a group refers to the ═O substituent.

The term “trifluoromethyl” as used herein alone or as part of a group refers to the —CF₃ radical.

The term “optional” or “optionally” as used herein means that the subsequently described event of circumstances may or may not occur, and that the description includes instances where said event or circumstance occurs and instances in which it does not.

Unless otherwise indicated, when a substituent is deemed to be “optionally substituted”, it is meant that the substituent is a group that may be substituted with one or more group(s) individually and independently selected from morpholinoalkanoate, cycloalkyl, aryl, heteroaryl, heterocyclyl, heteroalicyclic, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups, and the protected derivatives thereof.

For example, “optionally substituted aryl” means that the aryl radical may or may not be substituted and that the description includes both substituted aryl radicals and aryl radicals having no substitution.

The term “transition metal” as used herein refers to any element in the d-block of the Periodic Table of the elements. This corresponds to groups 3 (IIIB) to 12 (IIB) on the Periodic Table.

The term “ligand” in chemistry generally refers to an atom, ion, or molecule that bonds to a central metal, generally involving formal donation of one or more of its electrons. The metal-ligand bonding ranges from covalent to more ionic.

The term “carbene(s)” as used herein refers to an organic molecule containing a carbon atom with six valence electrons and having the general formula RR′C.

In one aspect, the present application is directed to a process for preparing a compound of formula (III), comprising reacting a compound of formula (I) with a compound of formula (II) in the presence of a transition metal catalyst or a precursor thereof,

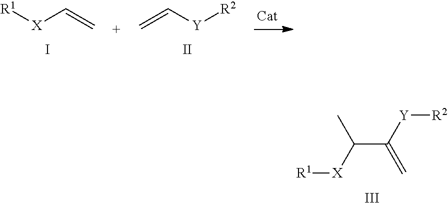

wherein,

X is independently selected from the atomic group consisting of Group 13 and Group 15-17 on the Periodic Table,

Y is independently selected from the atomic group consisting of Group 13 to 17 on the Periodic Table,

R¹ and R² are each independently selected from the group consisting of H, alkyl, alkenyl, aryl, arylalkyl, hydroxyl, alkoxyl, aroxyl, carbonyl, phosphonyl, halogen, amino, alkylamino, arylamino, mercapto, alkylthio and arylthio.

In some embodiments of the present application, X is O.

In some embodiments of the present application, R¹, and R² are each independently selected from the group consisting of alkyl, aryl and alkoxyl.

Examples of the compounds of formula (I) that may be used in the present application include, but are not limited to vinyl ethers, esters, thioethers, thioesters, fluoride, chloride, bromide, iodide, amines, phosphines and derivatives thereof.

Examples of the compounds of formula (II) that may be used in the present application include, but are not limited to vinyl ethers, esters, thioethers, thioesters, fluoride, chloride, bromide, iodide, amines, phosphines and the like, 1-hexene/1-octene (straight chain monoene), vinylcyclohexane, 4-methyl-1-pentene (branched chain monoene), styrene, allylbenzene (aromatic alkenes) and their substituted derivatives thereof, more highly substituted alkenes, and the like.

The methods of the present application may be used to catalytically couple two different monosubstituted alkenes in tail-to-tail manner to form a 1,1-disubstituted alkenes in one-pot. In some embodiments of the present application, the method is used to combine less reactive alkenes such as monoene or internal alkenes to build a more reactive one such as 1,1-disubstituted alkenes.

In some embodiments of the present application, the method is used to comprise two different alkenes in a single reaction chamber in the presence of a catalyst to form a branched allyl compound in nearly quantitative yield.

In one embodiment, the reaction may be intermolecular, i.e. the two reactants are not joined by a bond prior to the coupling reaction. In another embodiment, the reaction may be intramolecular.

The transition metal catalyst of the present application may include any catalytic transition metal and/or catalyst precursor as it is introduced into the reaction vessel and which may be, if needed, converted in situ into active form, as well as the active form of the catalyst which participates in the reaction. In some embodiments, the transition metal catalyst is provided in the reaction in a catalytic amount.

In some embodiments of the present application, the transition metal is selected from Groups 3 to 12 of the Periodic Table of Elements.

Exemplary transition metals that can be used in the present application include, but are not limited to, Scandium (Sc), Titanium (Ti), Vanadium (V), Chromium (Cr), Manganese (Mn), Iron (Fe), Cobalt (Co), Nickel (Ni), Copper (Cu), Zinc (Zn), Yttrium (Y), Zirconium (Zr), Niobium (Nb), Molybdenum (Mo), Technetium (Tc), Ruthenium (Ru), Rhodium (Rh), Palladium (Pd), Silver (Ag), Cadmium (Cd), Hafnium (Hf), Tantalum (Ta), Tungsten (W), Rhenium (Re), Osmium (Os), Iridium (Ir), Platinum (Pt), Gold (Au), Mercury (Hg), Rutherfordium (Rf), Dubnium (Db), Seaborgium (Sg), Bohrium (Bh), Hassium (Hs), Meitnerium (Mt), Darmstadtium (Ds), Roentgenium (Rg), and Ununbium (Uub).

In some embodiments of the present application, the transition metal is selected from Group 10 of the Periodic Table of Elements.

In some embodiments of the present application, the transition metal is selected from the group consisting of Nickel (Ni), Palladium (Pd) and Platinum (Pt). In some embodiments of the present application, the transition metal is Nickel (Ni).

The catalysts of the present application may also include heterogeneous catalysts that containing different forms of these above elements.

Ligands on the metal catalyst may include chelating ligands, such as (heterocyclic) carbene derivatives, and/or biscarbenes, bisheterocyclic carbenes, phosphines, amines, imines, arsines and derivatives thereof, including hybrids of the above.

In some embodiments of the present application, the ligand or metal bears a weakly or non-nucleophilic stabilizing ion, including but not limited to halogen, sulfonates, nitrates, nitrites and phosphonates. Weakly or non-nucleophilic stabilizing ions are preferred to avoid complicating side reaction of the counter ion, for example, attacking or adding to the electrophilic center of the substrates.

Exemplary amines that can be used in the present application include, but are not limited to, aliphatic amines, and aromatic amines. Exemplary aliphatic amines that can be used in the present application include, but are not limited to, primary amines, secondary amines, and tertiary amines. Exemplary aliphatic amines that can be used in the present application include, but are not limited to, methylamine, ethanolamine, dimethylamine, methylethanolamine, trimethylamine, aziridine, piperidine, N-methylpiperidine, and the like. Exemplary aromatic amines that can be used in the present application include, but are not limited to, aniline, o-toluidine, 2,4,6-trimethylaniline, anisidine, 3-trifluoromethylaniline, and the like.

In some embodiments of the present application, additional ligands may be included in the catalyst to obtain a stable complex.

The ligand can be added to the reaction mixture in the form of a metal complex, or added as separate reagent relative to the addition of the metal. The ligand, if chiral, can be provided as a racemic mixture or a purified stereoisomer. The ligands are commercially available or can be prepared by the methods similar to processes known in the art.

In some embodiments of the present application, the transition metal catalyst is provided in the reaction in a catalytic amount. In certain embodiments, that amount is in the range of <5 mol %, with respect to the limiting reagent, which may be either the compound of formula (I) or the compound of formula (II), depending upon which reagent is in stoichiometric insufficiency.

In some embodiments of the present application, the reaction is carried out in a solvent which is selected from the group consisting of aromatic hydrocarbons, aliphatic hydrocarbons, alicyclic hydrocarbons, halohydrocarbons, alcohols, ethers, esters, ketones, nitriles and diol derivatives, and ionic liquids such as imidazolium salts.

In some embodiments of the present application, the process is carried out with an ion exchange additive.

Exemplary aromatic hydrocarbons that can be used in the present application include, but are not limited to, benzene, toluene, xylene, and the like. Exemplary aliphatic hydrocarbons that can be used in the present application include, but are not limited to, pentane, hexane, heptane, octane, and the like. Exemplary alicyclic hydrocarbons that can be used in the present application include, but are not limited to, cyclohexane, cyclohexanone, methylcyclohexanone, and the like. Exemplary aliphatic hydrocarbons that can be used in the present application include, but are not limited to, pentane, hexane, heptane, octane, and the like. Exemplary halohydrocarbons that can be used in the present application include, but are not limited to, methylene chloride, chloroform, and the like. Exemplary alcohols that can be used in the present application include, but are not limited to, methanol, ethanol, isopropanol, and the like. Exemplary ethers that can be used in the present application include, but are not limited to, diethyl ether, methyl ethyl ether, propyl ether, propylene oxide, and the like. Exemplary esters that can be used in the present application include, but are not limited to, methyl formate, ethyl formate, butyl formate, pentyl formate, methyl acetate, ethyl acetate, propyl acetate, benzyl phenylacetate, and the like. Exemplary ketones that can be used in the present application include, but are not limited to, acetone, methylbutanone, methyl isobutyl ketone, and the like. Exemplary nitriles that can be used in the present application include, but are not limited to, acetonitrile, propionitrile, acrylonitrile, and the like. Exemplary diol derivatives that can be used in the present application include, but are not limited to, ethylene glycol monomethyl ether, ethylene glycol monoethyl ether, ethylene glycol monobutyl ether, and the like.

In some embodiments of the present application, the solvent is an aromatic hydrocarbon. In some embodiments of the present application, the solvent is selected from the group consisting of benzene, toluene and xylene. In some embodiments of the present application, the solvent is toluene.

Alternatively, the reaction can be carried out in the alkene substrates themselves (neat condition). Ionic liquid, such as imidazolium salts, can be also used as reaction medium.

In some embodiments of the present application, the process may be carried out optionally in a buffer to minimize the problems related to isomerization, oligomerization and polymerization. Examples of the buffer which can be used in the present application include but not limited to ammonium salt, phosphorous buffer, carbonates.

In another aspect, the present application is directed to a compound of formula (III)

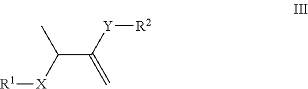

wherein,

X is selected from the atomic group consisting of Group 13 and Group 15-17 on the Periodic Table,

Y is independently selected from the atomic group consisting of Group 13 to 17 on the Periodic Table,

R¹ and R² are each independently selected from the group consisting of H, alkyl, alkenyl, aryl, arylalkyl, hydroxyl, alkoxyl, aroxyl, carbonyl, phosphonyl, halogen, amino, alkylamino, arylamino, mercapto, alkylthio and arylthio.

In some embodiments of the present application, X is selected from the atomic group consisting of Group 13 and Group 15 to 17 on the Periodic Table,

In some embodiments of the present application, Y is selected from the atomic group consisting of Group 13 to 17 on the Periodic Table,

In some embodiments of the present application, R¹ and R² are each independently selected from the group consisting of H, alkyl, alkenyl, aryl arylkyl, hydroxyl, alkoxyl, aroxyl, carbonyl, phosphonyl, halogen, amino, alkylamino, arylamino, mercapto, alkylthio and arylthio.

The following examples are provided by way of illustration and not by way of limitation.

### EXAMPLES

The active catalyst was generated by using a transition metal with a (heterocyclic) carbene ligand and a hydride precursor, with a general structure of [carbene-M-H]Z.

The transformation was achieved by adding the corresponding alkenes to the catalyst solution, stirring at room temperature for 24 hrs under nitrogen atmosphere.

A buffer may be used to minimize the problems related to isomerization, oligomerization and polymerization.

An ion exchange additive may be used to substitute or exchange the ion Z for reaction rate improvement

Typical procedure for the in situ catalyst generation:

Under a nitrogen/inert atmosphere, to a solution of a carbene (5 mol %) and Ni(cod)₂ (5 mol %) in 2 mL toluene in a typical round bottom flask equipped with a magnetic stir bar, 1-octene (10 mol %), triethylamine (15 mol %), p-anisaldehyde (5 mol %) and silyl triflate (10 mol %) were added sequentially at room temperature. The catalyst was generated after 30 mins of stirring at room temperature using normal bench-top apparatus. In situ catalyst generation can be done alternatively using a compound with a general formula of benzyl or allyl-Z to replace the carbonyl compounds, where Z equal to leaving group. Also the catalyst could be generated by oxidative addition using the corresponding ionic liquid and Ni(cod)₂.

Typical branched allyl compounds preparation procedure:

The two different alkene substrates can be added to the catalyst mixture after the catalyst generation. Keep on stirring for another 24 hrs at rt and normal pressure on bench top, work up by filtering it through a pad of silica gel and concentrate in vacuum. (e.g. Commercially available alkyl vinyl ether, 100% conversion, quantitative yield based on the alkyl vinyl ether, with the corresponding branched allyl ether as exclusive isomer).

Following the above general procedures, various branched allyl ethers were synthesized from the corresponding starting materials and the characterization data thereof are provided. In the following Examples, no other isomers were observed in significant amount unless otherwise indicated. The yields were based on vinyl ether and average of at least two runs unless otherwise indicated.

### Example 1

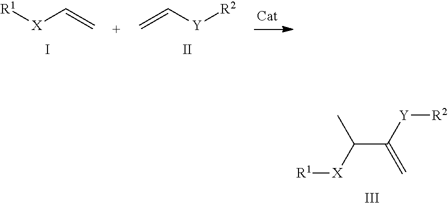

Catalyst generation: Ni(cod)₂ and IPr (0.05 mmol, 5 mol % each) were added to an oven-dried test tube equipped with a stir bar in glove box. After sealed with a septum and brought out of the glove box, it was connected to a N₂ line. The mixture was dissolved in 2 mL degassed toluene and stirred at room temperature for 1 h. 1-octene (10 mol %), NEt₃ (15 mol %), p-anisaldehyde (5 mol %), TESOTf (10 mol %) were then added sequentially and stirred 15 mins at room temperature.

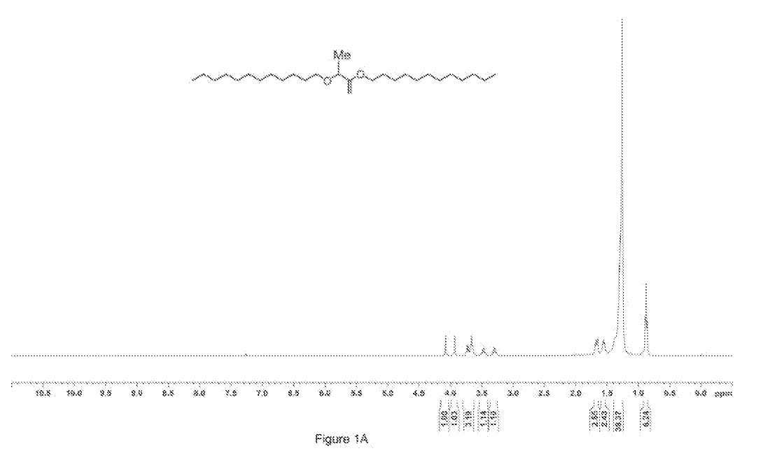

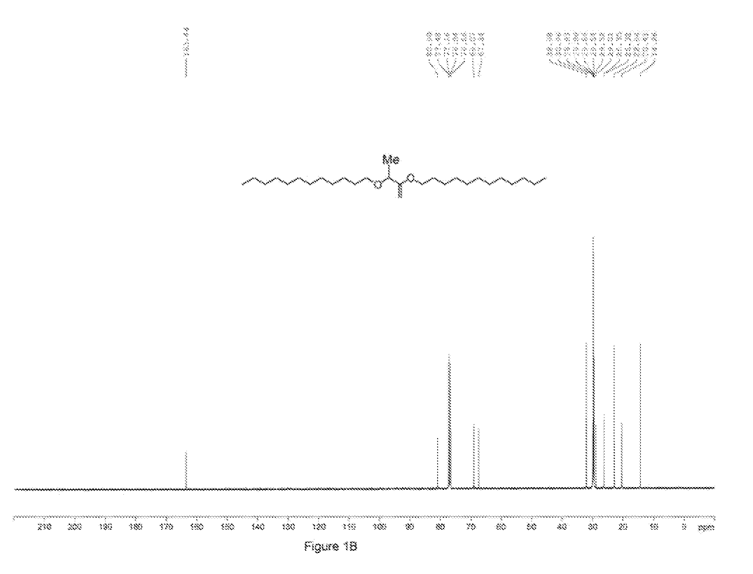

The alkyl vinyl ethers (1.0 mmol each) were added to the in situ generated catalyst mixture [(5 mol % “[IPr—Ni—H]OTf”) and 15 mol % NEt₃ in 2.0 mL toluene, see below] at room temperature and stirred for 24 hrs. The desired branched allyl ether was isolated by typical silica gel column chromatography. No other isomers were found in the reaction. The ¹H- and ¹³C-NMR spectra of the desired branched allyl ether were shown in FIGS. 1A and 1B, respectively.

All of the above patents, patent application publications, patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the application have been described herein for purposes of illustration, various modifications or variations may be made by those skilled in the art without deviating from the spirit and scope of the application.

What the claimed is:

1. A process for preparing a compound of formula (III), comprising reacting a compound of formula (I) with a compound of formula (II) in the presence of a transition metal catalyst or a precursor thereof, 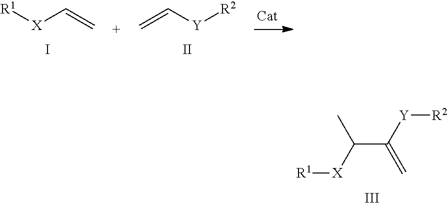
wherein,
X is independently selected from the atomic group consisting of Group 13 and Group 15-17 on the Periodic Table,
Y is independently selected from the atomic group consisting of Group 13 to 17 on the Periodic Table,
R¹ and R² are each independently selected from the group consisting of H, alkyl, alkenyl, aryl, arylalkyl, hydroxyl, alkoxyl, aroxyl, carbonyl, phosphonyl, halogen, amino, alkylamino, arylamino, mercapto, alkylthio and arylthio.

2. A process of claim 1, wherein X and Y are each independently selected from the group consisting of O, S, N, P.

3. A process of claim 1, wherein R¹, and R² are each independently selected from the group consisting of alkyl, aryl, alkoxyl and derivatives thereof.

4. A process of claim 1, wherein the compound of formula (I) is selected from the group consisting of vinyl ethers, esters, thioethers, thioesters, fluoride, chloride, bromide, iodide, amines, phosphines and derivatives thereof.

5. A process of claim 1, wherein the compound of formula (II) is selected from the group consisting of straight chain monoenes, branched chain monoenes, aromatic alkenes, and their substituted derivatives.

6. A process of claim 1, wherein the transition metal is selected from Groups 3 to 12 of the Periodic Table of Elements.

7. A process of claim 1, wherein the transition metal catalyst comprises a ligand which is selected from the group consisting of carbenes, heterocyclic carbenes, biscarbenes, bisheterocyclic carbenes, phosphines, amines, imines, arsines and derivatives thereof.

8. A process of claim 1, wherein the amount of the transition metal is <5 mol %, with respect to a limiting reagent, which is either the compound of formula (I) or the compound of formula (II), depending upon which reagent is in stoichiometric insufficiency.

9. A process of claim 1, wherein the process is carried out in a solvent which is selected from the group consisting of aromatic hydrocarbons, aliphatic hydrocarbons, alicyclic hydrocarbons, halohydrocarbons, alcohols, ethers, esters, ketones, nitriles and diol derivatives, and ionic liquids.

10. A process of claim 1, wherein the process is carried out in a buffer.

11. A process of claim 1, wherein the process is carried out with an ion exchange additive.

12. A process of claim 1, wherein the ligand or metal bears a weakly or non-nucleophilic stabilizing ion which is selected from the group consisting of halogen, sulfonates, nitrates, nitrites, phosphates and phosphonates.

13. A process of claim 1, wherein the ligand is chiral and is provided as a racemic mixture or a purified stereoisomer.
